(19) United States
(12) Patent Application Publication
(10) Pub. No.: US 2007/0224214 A1
(43) Pub. Date: Sep. 27, 2007

(54) Composition Exerting Physiological Activity Via Biological Immune Mechanism

(75) Inventor: Tomihisa Ota, Kanazawa-shi (JP)

Correspondence Address: MARK D. SARALINO (GENERAL);RENNER, OTTO, BOISSELLE & SKLAR, LLP, 1621 EUCLID AVENUE, NINETEENTH FLOOR, CLEVELAND, OH, 44115-2191, US

(73) Assignees: SUNDORY CO., LTD., Osaka (JP); KYOWA ENGINEERING CO., LTD., Tokyo (JP)

(21) Appl. No.: 10/571,849

(22) PCT Filed: Sep. 6, 2004
(86) PCT No.: PCT/JP04/12957

§ 371 Date: Nov. 14, 2006

(30) Foreign Application Priority Data
Sep. 17, 2003 (JP) 2003-325337

(51) Int. Cl. A61K 36/06

(52) U.S. Cl. 424/195.15

(57) ABSTRACT

It is intended to provide a low-molecular weight component (or fraction) originating in Agaricus Blazei Muril which is excellent in digestion and absorption properties in vivo and exerts a physiological activity via the immune mechanism in vivo, and a food material or a drug material. Namely, a composition exerting a physiological activity via the immune mechanism in vivo and containing a hot water extract of Agaricus Blazei Muril. This immune mechanism is mediated by immunocompetent cells which can be selected from a group consisting of macrophages, T cells and killer cells. In a typical case, the physiological activity is a tumor-suppressive effect and the tumor is sarcoma.

### TECHNICAL FIELD

The present invention is directed to a composition, which contains an extract of Agaricus blazei muril, and exerts a physiological activity though the immune mechanism of the living body.

### BACKGROUND ART

As the wish for fulfillment and maintaining health increases, many people generally tend to take health foods and health food supplements. People pay attention to naturally occurring materials containing active ingredients (especially, those having anti-tumor effects) without unwanted side effects, because many chemical therapeutic drugs or synthetic compounds exhibit many side effects along with anti-tumor activities. Mushroom, Agaricales fruit body and the like are known as a naturally occurring material which has an active ingredient.

Many polysaccharides, which have an anti-tumor activity, have been isolated from Mushroom (Non-patent document 1: Hamono J et al. (1978), Cancer Res. 38:3080-3085; Mizuno T et al. (1992), Biosci. Biotechnol. Biochem. 56:347-348).

The Mushroom, which is generally called Agaricus, belongs to the family Agaricaceae of the division Basidiomycota, and is referred to by the botanical name “Agaricus blazei Murill” and the Japanese name “Kawariharatake”. Agaricus (hereinafter, generally referred to as Kawariharatake, ABM, or agaricus) has been traditionally used as a medicament in the Piedade region in Sao Paulo, Brazil. It is said that Agaricus has a variety of immunostimulatory activities, cancer prevention effects, tumor growth suppression effects, and the like. Currently, it is widely provided for internal use as health food. The terms “cancer” and “tumor” are used as being exchangeable herein.

Polysaccharides contained in Kawariharatake include β-1,6-glucopyranosyl residues and have antitumor activity against Sarcoma 180 (Non-patent document 2: Ebina T et al. (1986), Jpn. J. Cancer Res 77:1034-1042).

Extracts from Kawariharatake include (1→4)-α-D-glucan having (1→6)-β branched chain, and have natural killer cell activation activity and selective anti-tumor activity mediated through apoptosis (Non-patent document 3: Fujimiya Y et al. (1998), Cancer Immunol Immunother 46:147-159).

Peptide glycans from Kawariharatake have a direct cytotoxic activity against Meth A tumor cells in a double implanted tumor system and an indirect immune enhancement activity in tumor-bearing mice (Non-patent document 4: Ebina T et al. (1998), Biotherapy 11:259-265).

Polysaccharides contained in Kawariharatake change the percentage of spleen Thy1,2-,L3T4 positive cells in a T cell subset of mice (Non-patent document 5: Mizuno M et al. (1998), Biosci. Biotechnol. Biochem. 62:434-437).

These reports suggest that polysaccharides contained in Kawariharatake have cytotoxic activity against tumor cells through an immunomodulatory activity.

However, prior art documents including the above documents, concern about high molecular weight materials derived from Kawariharatake. Most of the document are silent as to low molecular weight materials (or fractions) having a physiological activity from Kawariharatake.

Non-patent document 1: Hamono J et al. (1978), Cancer Res. 38:3080-3085; Mizuno T et al. (1992), Biosci. Biotechnol. Biochem. 56:347-348).

Non-patent document 2: Ebina T et al. (1986), Jpn. J. Cancer Res 77:1034-1042).

Non-patent document 3: Fujimiya Y et al. (1998), Cancer Immunol Immunother 46:147-159.

Non-patent document 4: Ebina T et al. (1998), Biotherapy 11:259-265.

Non-patent document 5: Mizuno M et al. (1998), Biosci. Biotechnol. Biochem. 62:434-437.

### DISCLOSURE OF THE INVENTION

### Problems to be Solved by the Invention

It is believed that macromolecules generally are not digested and absorbed well compared to low molecular materials. The present invention aims to provide a low molecular ingredient (or fraction) having a physiological activity, which is digested and absorbed well in the living body.

### MEANS FOR SOLVING THE PROBLEMS

The inventors have completed the subject invention by analyzing the action mechanism of growth suppression effects on the tumor cells implanted into mice, that is exerted by a hot water extract and the fraction thereof.

The present invention is directed to a composition for exerting a physiological activity through the immune mechanism of the living body, the composition comprises a hot water extract of Agaricus blazei muril.

The immune mechanism can be mediated by immunocompetent cells.

The immunocompetent cells can be selected from a group consisting of macrophages, T cells and killer cells.

The immunocompetent cells can be macrophages.

The physiological activity can be a tumor-suppressive effect.

The tumor can be sarcoma.

The sarcoma can be Sarcoma 180 or Meth A fibrosarcoma.

The physiological activity can have the effect of the prolongating of life.

Preferably, the composition further comprises a pharmaceutically acceptable carrier.

The composition can be in the form selected from a group consisting of powder, liquid, tablet, capsule, and pellet.

The term “immunocompetent cells” used herein refers to cells responsible for an immune response known to those skilled in the art, and include lymphocytes divided into B cells mediating a bodily immune system, and T cells mediating cellular immune system; accessory cells such as macrophages, Langerhans cells, dendrite cells; NK cells (natural killer cells); LAK cells (lymphokine activated killer cells); and cells inducing an antibody dependent cellular injury, and the like. T cells include helper cells and suppressor T cells concerning the control of an immune response, killer T cells destroying target cells, and T cells involved in delayed hypersensitivity. B cells include antibody secreting cells which are differentiated from B cells due to activation by antigen and T cells.

The hot water extract of Kawariharatake can contain the main chromatographic elution fraction having a molecular weight of 100 to 2000 which is obtained by the steps of extracting a fruit body of Kawariharatake with hot water, dialyzing the extract, and subjecting the dialysate outside the dialysis bag to chromatography.

Alternatively, the hot water extract of Kawariharatake can contain an effective ingredient in a dialysate outside the dialysis bag, obtained by the steps of extracting a fruit body of Kawariharatake with hot water, adding ethanol to the resultant extract to thereby divide into a precipitate and a supernatant by centrifugation, adding ethanol to the supernatant to thereby divide into a precipitate and a supernatant by centrifugation, and dissolving the precipitates in distillated water, and dialyzing the solution.

The hot water extract of Kawariharatake optionally can be in the form of a blend with a pharmaceutically acceptable carrier. Such a pharmaceutically acceptable carrier is known to those skilled in the art, and comprises the following without being limited thereto: Ringer's solution, Hank's solution, or buffer such as buffered saline; fatty acid such as sesame oil, synthetic fatty acid ester such as ethyl oleate or triglyceride; sugars such as lactose, sucrose, mannitol, sorbitol: plant derived starch such as maze, wheat, rice, potato; cellulose such as methyl cellulose, hydroxy-propyl-methyl cellulose; sodium carboxy-methyl cellulose; rubber such as gum arabic, tragacanth; protein such as gelatin, collagen; cross-linked polyvinylpyrrolidone, agar, alginic acid or the salts thereof.

### EFFECTS OF THE INVENTION

Low molecular ingredients (or fraction) having superior physiological activities, which are derived from Kawariharatake and digested and absorbed well in the living body, are provided. These low molecular ingredients (or fractions) are useful as a food material or a drug material.

### BRIEF DESCRIPTION OF THE DRAWINGS

FIG. 1 shows a growth curve of tumors implanted in mice. Tumor suppressing effects of the composition according to the present invention are indicated.

FIG. 2 shows growth (represented by the volume of tumor) of tumors implanted in mice. A indicates growth of tumor in nude mice, and B indicates growth of tumor in normal mice.

FIG. 3 is a photograph indicating a comparison among tumor masses in nude mice administered with the composition of the present invention.

FIG. 4 shows growth of tumors (represented by tumor volume) in mice, which is selectively removed of NK cells by I.V. injection of anti-asialo antibody.

FIG. 5 shows growth of tumors (represented by tumor volume) in mice, in which macrophages are selectively inhibited by I.V. injection of 2-chloroadenosine.

FIG. 6 is a drawing indicating a comparison among tumor mass weights in mice of each group in the experiment in FIG. 4.

FIG. 7 is a photograph indicating a comparison among tumor sizes in mice of each group in the experiment in FIG. 4.

FIG. 8 is a photograph indicating a comparison among tumor sizes in mice of each group in the experiment in FIG. 5. In the figure, 2-chloroadenosine indicates 2-chloroadenosine treatment.

FIG. 9 is a drawing indicating a comparison among tumor masse weights at day 35 in mice of each group in the experiment in FIG. 5.

FIG. 10 is a drawing indicating a comparison among tumor mass volumes at day 35 in mice of each group in the experiment in FIG. 5.

FIG. 11 is a drawing indicating the effects of the prolongation of life of the subject composition in mice.

FIG. 12 is a drawing indicating the transitions of tumor sizes in each mouse in the experiment in FIG. 11.

FIG. 13 is a drawing indicating the transitions of body weights in each mouse in the experiment in FIG. 11.

FIG. 14 is a drawing indicating the amount of drinking in each mouse in the experiment in FIG. 11.

FIG. 15 is a drawing indicating the effects of the prolongation of life of the subject composition in mice.

FIG. 16 shows growth of tumors implanted in mice. Tumor suppressing effects of the composition according to the present invention are indicated.

FIG. 17 is a drawing indicating the transitions of body weights in each mouse in the experiment in FIG. 16.

FIG. 18 is a photograph indicating the transitions of tumor sizes in each mouse in the experiment in FIG. 16.

### BEST MODE FOR CARRYING OUT THE INVENTION

The method for manufacturing the composition, which exerts a physiological activity through the immune system of the living body of the present invention, is described hereinafter.

Kawariharatake extract of the present invention is prepared by extracting Kawariharatake material with a solvent. The Kawariharatake material is typically a naturally occurring or cultured fruit body of Kawariharatake. Cultured mycelium of Kawariharatake in a culture tank or the like can also be used. Normally, Kawariharatake is used after washing followed by drying. Commercially available dried fruit body is also used conveniently. Normally, dried Kawariharatake is pulverized according to a standard method, and used as a starting material for extraction.

The Kawariharatake extract of the present invention can be obtained by conducting an extraction procedure after adding a solvent to the dried fruit body or a powder thereof. In general, the dried fruit body, or the powder thereof, is mixed with a solvent of 2 to 10 times the weight thereof to conduct extraction. As the solvent, water, ethanol, propanol, butanol, acetone, 1,3-butylene glycol, ethyl acetate, hexane, methylene chloride, methanol, or a mixture thereof is used. Typically, water is used to prepare an Kawariharatake extract.

Hot water extraction procedure of Kawariharatake is conducted by mixing the fruit body or powder of Kawariharatake with 5 to 10 times of the weight of water, and followed by heat-extracting or heat-refluxing the mixture for 1 to 3 hours. This hot water extraction from Kawariharatake can be performed using a residue previously extracted with hot water. The solution extracted with hot water thus obtained (also herein sometimes referred to ABMK-WW) is dried by a method known to those skilled in the art such as lyophilization, spray-drying, or the like to obtain a dried product (hereinafter, referred to as dried product A). This dried product is mixed with 5 to 20 times the weight thereof of water. Then, the solution is put into a dialysis tube and dialyzed for 10 to 15 hours with several times the amount thereof of distilled water. The obtained dialyzate (external fluid; also herein sometimes referred to ABMK-WLM or WLM) is lyophilized to obtain a dried product (hereinafter, referred to as dried product C) of hot water extract of Kawariharatake.

Then, the solution remaining in the dialysis tube is further dialyzed against running water for 20 to 40 hours and dialyzed twice against distilled water for a few hours each time and a dried product of the solution remaining in the dialysis tube (referred also herein sometimes referred to ABMK-WHM or WHM) is obtained as described above. Thus, the dried product (hereinafter, referred to as dried product B) capable of exerting a physiological activity through an immune mechanism of the living body can be obtained.

Next, the obtained dried product C is dissolved in about ten times the weight thereof of distilled water. Gel chromatography is performed with distilled water as an eluent to obtain fractions of 20 mL. From the obtained fractions, a fraction or fractions in the middle of the elution peak, which has a molecular weight of about 100-2000 Da by gel filtration, is also a fraction capable of exerting a physiological activity through an immune mechanism of the living body.

These fractions were analyzed further using reverse-phase chromatography, which uses ODS (octadecyl silanated silica gel), ion-exchange chromatography using DEAE-TOYOPEARL 650, or the like, and confirmed to include a plurality of ingredients such as arginine, lysine, mannitol, and the like.

The solution extracted with hot water, obtained by the above-described method, is mixed with an equal amount of ethanol. The mixture is centrifuged to separate a precipitate from a supernatant. The obtained supernatant is further mixed with 1 to 3 volumes of ethanol thereof. The mixture is further centrifuged to obtain a precipitate. The precipitate obtained is dissolved in distilled water and the solution obtained, is dialyzed. The dialyzate obtained is also a low molecular weight fraction which is a fraction capable of exerting a physiological activity through an immune mechanism of the living body of the present invention.

The thus obtained fraction containing a hot water extract of Kawariharatake can be used in manufacturing medical formulations along with various carriers. This fraction containing a hot water extract of Kawariharatake can be utilized per se, or by combining with other food materials, as a health food.

Typically, the composition of the present invention can be administered orally along with a physiologically compatible pharmaceutical carrier (For example, saline, buffered physiological saline, dextrose, and water, or the like). The composition of the present invention can be ingested alone or in combination with other drug or food material.

The composition of the present invention can be administered orally or parenterally. Parenteral administration is accomplished via intravenous, intramuscular, intra-abdominal or intranasal administration. The details of formulation and administration of the pharmaceutical composition according to the present invention can be performed in accordance with descriptions in a textbook in the field of art, for example, “REMINGTON'S PHARMACEUTICAL SCIENCES” (Maack Publishing Co., Easton, Pa.).

The composition for oral administration can be formulated as a composition including a pharmaceutically acceptable carrier well known in the art, in a prescription form suitable for ingestion. Such a carrier allows the composition obtained to be formulated as a tablet, pill, sugar-coated pill, capsule, liquid, gel, syrup, slurry, suspension, or the like, suitable for ingestion by patients.

The composition of the present invention includes Kawariharatake extract contained therein in an amount effective for exerting a physiological activity. Those skilled in the art will thoroughly understand and recognize the “pharmaceutically effective amount” that means the amount of hot water extract of Kawariharatake effective to exert an intended physiological activity in vivo through an immune mechanism of the living body. Accordingly, a pharmaceutically effective amount is a sufficient amount for exerting a physiological activity through an immune mechanism of the living body.

An example of a useful assay to confirm the “pharmaceutically effective amount” is using an animal model lacking an immune mechanism and a control animal, and comparing the tumor growth therebetween after implanting the same kind of tumor thereto and then administering the Kawariharatake extract. Such an animal model is well known to those skilled in the art. The amount of the Kawariharatake extract which is actually ingested depends on the health conditions, or the like of the individual to which the extract is applied and may be optimized so that a desirable effect can be achieved. It is a routine process for those skilled in the art to determine a pharmaceutically effective amount.

Initially, the pharmaceutically effective amount can be evaluated by an in vitro assay by cell culture or appropriate animal model. Then, using such information, an effective amount for administration in a human can be determined. The pharmaceutically effective amount, generally, can be in the range of about 1 mg/kg body weight/day to about 500 mg/kg body weight/day, preferably about 5 mg/kg body weight/day to about 200 mg/kg body weight/day.

Further, the above described fraction exerting a physiological activity through an immune mechanism of the living body can be mixed with one or more selected food materials in an amount sufficient for exerting its function. The one or more selected food materials are mixed with the fraction having an immune activation activity in a form known to those skilled in the art, usually, powder form. The mixture can be served as a liquid food product depending on its utility or on preference. Alternatively, the mixture may be formed as capsules such as hard capsules or soft capsules, tablets, or pills, or may be formed into a powdery, granular, tea-leaf, tea-bag, or candy form.

Hereinafter, the present invention will be further described by way of examples. The following examples are merely illustrative and do not limit the present invention.

### EXAMPLES

### Example 1

### Preparation of Kawariharatake Extract

(1) The above described dried product A was used as a Kawariharatake hot water extract. This was obtained by extracting a dried fruit body of Kawariharatake (Kyowa's Agaricus Mushroom (available from Kyowa Engineering Co., Ltd.)) with boiling water, removing residue by centrifugation, at 1800×g, for 10 minutes followed by lyophilization. This is dissolved in purified water at a concentration of 3.7 mg/ml to form Sample I, and at a concentration of 8 mg/ml to form Sample II.

(2) Distilled water (2 L) was added to 300 g of Kyowa's Agaricus Mushroom, and the mixture was heated to reflux for two hours. The solution obtained was filtered to separate a filtrate (a solution extracted with hot water) and a residue. Again, 2 L of distilled water was added to the residue and the mixture was heated to reflux for another two hours to perform hot water extraction and a filtrate was obtained. Further, the same procedure was repeated one more time. The filtrates obtained were lyophilized together to obtain dried product A (153 g: extraction rate of 51%).

Distilled water (500 mL) was added to 50 g of dried product A and the mixture was put into a dialysis tube (Spectra/Por Membrane 50×31, inner diameter of 8 mm and length of 30 cm, FE-0526-65). The mixture was dialyzed against 3 L of distilled water for 12 hours. The dialyzate obtained outside the tube was lyophilized to obtain dried product C (27 g: extraction rate of 53%). The solution remaining in the dialysis tube was further dialyzed against running water for 30 hours, and then dialyzed twice against distilled water (four hours each time, for a total of 8 hours). Thereafter, the solution remaining in the dialysis tube was lyophilized to obtain dried product B (11 g: extraction rate of 22%). Subsequently, 3 g of dried product C was dissolved in 30 mL of distilled water and chromatography using TOYOPEARL HW40C (inner diameter of 40 mm and length of 420 mm) was performed. The eluent was entirely distilled water. For each fraction, 20 ml of the aliquots were taken to obtain fractions 1 to 30. These fractions were divided into the following five groups with reference to results of thin-layer chromatography analysis. The dried weights were as follows: fractions 1 to 11 (75 mg, 2.5%); fractions 12 to 15 (920 mg, 30.7%); fractions 16 to 17 (1570 mg, 52.3%); fractions 18 to 19 (270 mg, 9%); and fractions 20 to 28 (97 mg, 3.2%).

Infrared radiation (IR) absorption spectrum data of fraction 16 (hereinafter, sometimes referred to as 1SY-16) was as follows.

Fraction 16: IR (KBr) 3390, 3325, 3285, 2940, 2920, 1641, 1634, 1622, 1615, 1600, 1595, 1405, 1394, 1084, 1020: molecular weight (estimated by gel filtration) 100-2000 Da

(3) Hot water extraction similar to that described above was performed to obtain 6 L of a combined filtrate (a solution extracted with hot water). The filtrate was concentrated under reduced pressure to 1 L, and 1 L of ethanol was added thereto and mixed, followed by centrifugation to obtain a precipitate and a supernatant. Ethanol (3 L) was further added to the supernatant and mixed, and the mixture was centrifuged to obtain a precipitate, and the precipitate was dissolved in distilled water and dialyzed. The external dialyzate obtained was lyophilized to obtain a powder (hereinafter sometimes referred to as ABMK-22).

### Example 2

### Study of Action Mechanism of Tumor Growth Suppression Effects by Kawariharatake Extract

1. Expression of the Physiological Activity of Kawariharatake Extract Through T Cells.

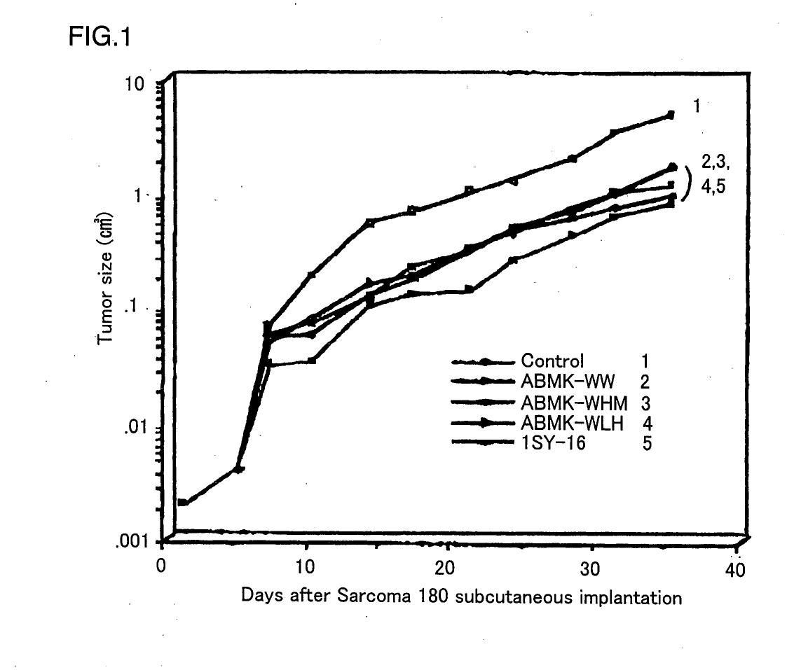

We have recognized tumor growth suppression effects when the above described hot water extract of Kawariharatake and the fractionated fraction thereof is administered orally to mice which are implanted by Sarcoma 180. FIG. 1 shows one example thereof. The graph shown in FIG. 1 is a growth curve of Sarcoma 180 implanted subcutaneously. In the figure, the horizontal line (X-axis) indicates the number of days after Sarcoma 180 subcutaneous implantation. The vertical line (Y-axis) indicates a tumor size (volume, cm³). In the figure, reference numeral 1 indicates the control group, and reference numerals 2 to 5 indicate results in groups which are administered with Kawariharatake and the fractionated fraction thereof.

It is generally known that when a tumor growth curve is represented by a logarithmic plot, absolute number of tumor cells decreases significantly concurrently with administration of a chemotherapeutic in a curve pattern. Thereafter, it is recognized that the parallel growth of tumor cells occurs when compared to those of the control group. On the contrary, it is known that in the event that tumor cells are treated by a material that exhibits tumor suppressive action through an immune mechanism of the living body, the growth curve of tumor cells become gradually flat one to two weeks after administration thereof. Since the growth curve indicated in FIG. 1 belongs to the latter, it was considered that the hot water extract of Kawariharatake, or fractionated fraction thereof, exerts a tumor growth suppressive action due to an activated immune system mediated by various biomolecules. This was supported by the fact that events such as body weight loss, which is usually caused by side-effects, have not been observed during these experiments.

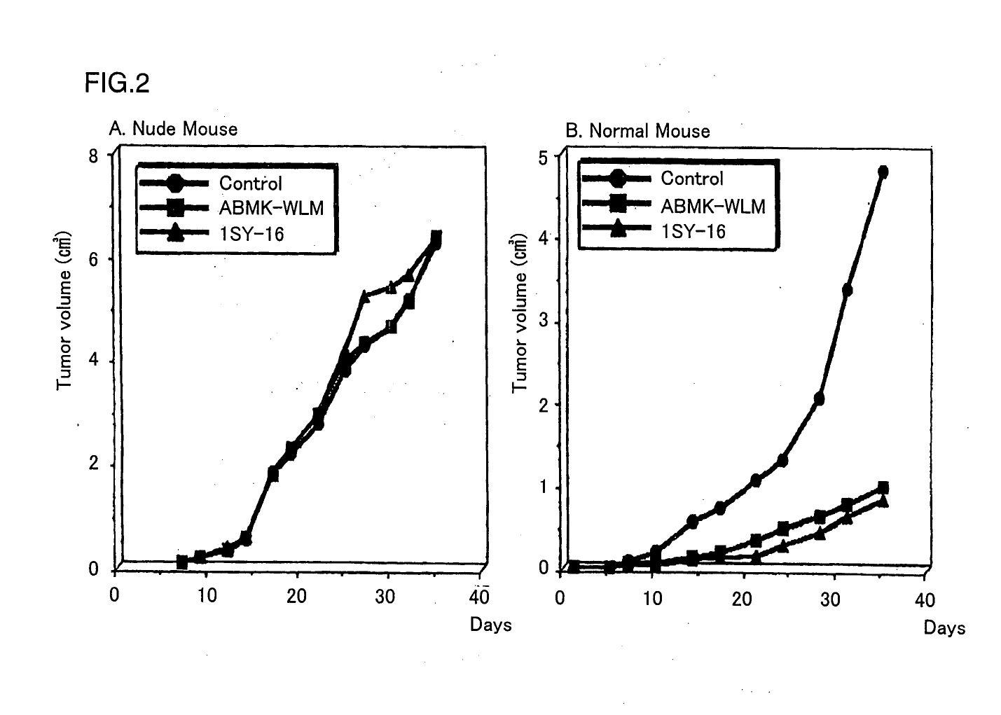

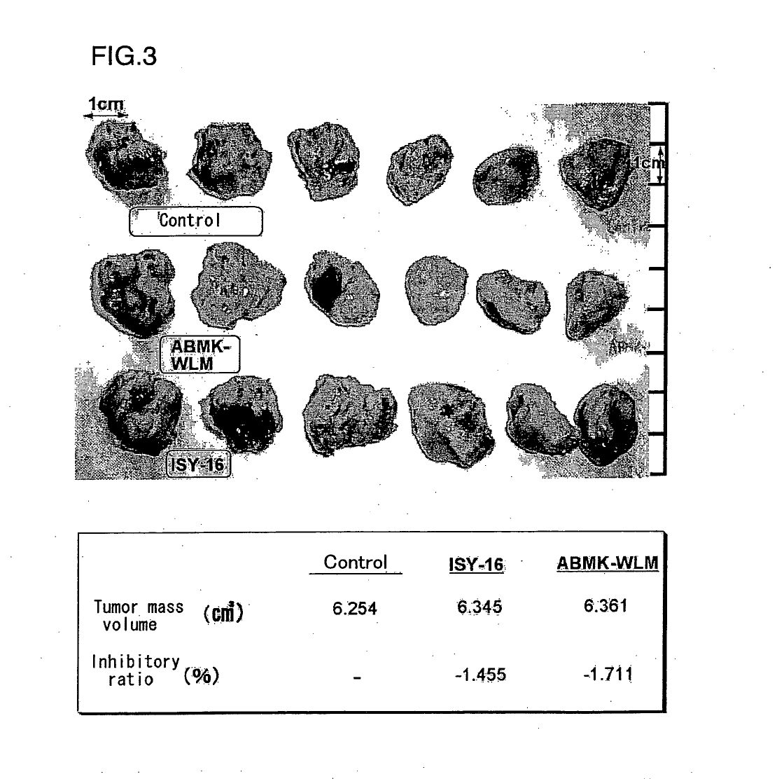

Due to this fact, we conducted an assay, similar to the above assay, using nude mice ICR/JCL-nunu which lack T cells function, and administering a hot water extract of Kawariharatake or fractionated fraction thereof orally to these animals (corresponding to about 100 mg ABMK-WW/kg/day). Results thereof are indicated in FIG. 2. In the figure, A indicates results in nude mice, and B indicates results in normal-mice. In the graph of the figure, the horizontal line (X-axis) indicates the number of days after Sarcoma 180 implantation, and the vertical line (Y-axis) indicates tumor volume (cm³). Further, the square legend shows results in the ABMK-WLM (outer dialysate) administered group, and the triangle legend shows results in 1SY-16 administered group, respectively. The circle legend shows results in the control group. Unless specified otherwise herein, six mice were used in one experimental group. As a result, as indicated in FIG. 2A, ABMK-WLM and 1SY-16 did not show tumor growth suppressive effects at all, contrary to results when using mice having normal T cells (FIG. 2B). Based on these results, it was believed that hot water extract or fractionated fraction thereof of Kawariharatake exerts a physiological activity, i.e. a tumor suppressive action through murine T cells directed to Sarcoma 180. Tables 1 and 2 indicate changes in tumor sizes (table 1), tumor weight and tumor size at day 35, and inhibitory ratio (table 2) in each group of nude mice in the experiment shown in FIG. 2, respectively. FIG. 3 is a photograph indicating a comparison of tumor mass in each group of nude mice.

TABLE 1

| Days after | Control | 1SY-16 | ABMK-WLM |
|---|---|---|---|
| injection | (cm³) | (cm³) | (cm³) |
| 7 | 0.090 | 0.088 | 0.089 |
| 9 | 0.192 | 0.190 | 0.191 |
| 12 | 0.331 | 0.359 | 0.322 |
| 17 | 1.796 | 1.789 | 1.749 |
| 22 | 2.754 | 2.925 | 2.923 |
| 27 | 4.264 | 5.204 | 4.321 |
| 30 | 4.625 | 5.393 | 4.627 |
| 35 | 6.264 | 6.345 | 6.361 |

TABLE 2

| Test | Tumor | Inhibitory | Tumor | Inhibitory |
|---|---|---|---|---|
| sample | weight(g) | ratio(%) | size(cm³) | ratio(%) |
| control | 3.76 | — | 6.254 | — |
| 1SY-16 | 3.89 | −3.46 | 6.345 | −1.434 |
| ABMK-WLM | 3.84 | −2.08 | 6.361 | −1.711 |

2. Physiological Activity Exertion of Kawariharatake Extract Through NK Cells or Macrophages.

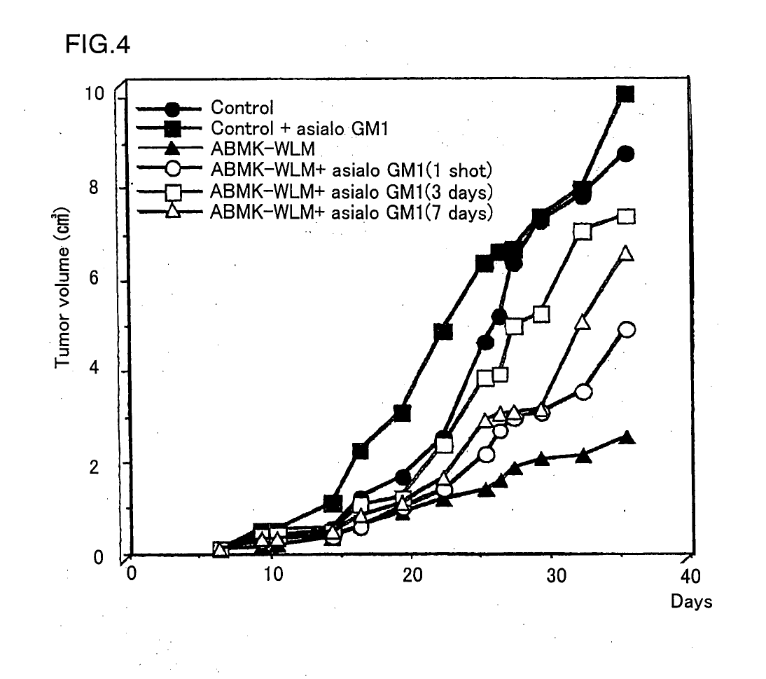

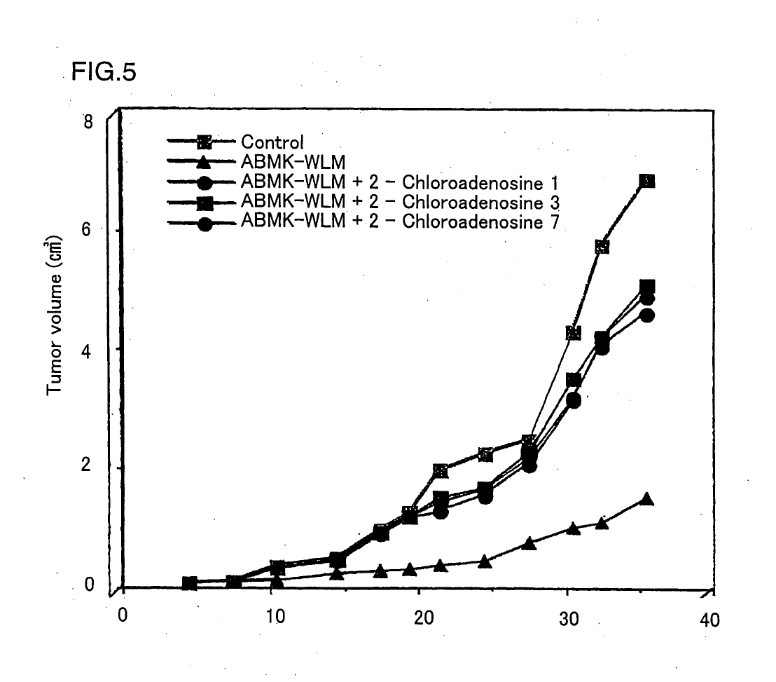

Further we paid attention to NK cells and macrophages among cells or agents involved in immune mechanisms which govern protection of the living body, physiological activity thereto of Kawariharatake extract or fractionated fraction thereof was investigated. An activity assay similar to the above described 1 was conducted using the ICR/JCL mouse administered with anti-asialo antibody, which is known to remove NK cells selectively, through the tail vein, and the ICR/JCL mouse administered with 2-chloroadenosine administered through intra vein, which is known to being selectively toxic to macrophages. Results are shown in FIG. 4 and FIG. 5.

FIG. 4 shows results of the experiment using the ICR/JCL mouse treated with anti-asialo antibody (in the figure, shown as asialo GM1). FIG. 5 shows results of the experiment using the ICR/JCL mouse treated with 2-chloroadenosine. As can be seen from FIGS. 4 and 5, tumor volumes in anti-asialo antibody treated mice, and 2-choloadenosine treated mice (in FIG. 4, shown as a closed square, open circle, open square, open triangle, and in FIG. 5, shown as a closed circle and closed square) were significantly larger compared to tumor volumes in ABMK-WLM administered mice without anti-asialo antibody treatment and 2-chloroadenosine treatment (in FIGS. 4 and 5, shown as a closed triangle). Based on these results, it is considered that the hot water extract or fractionated fraction thereof exerts the physiological activity thereof through NK cells and macrophages in mice thereby suppressing tumors.

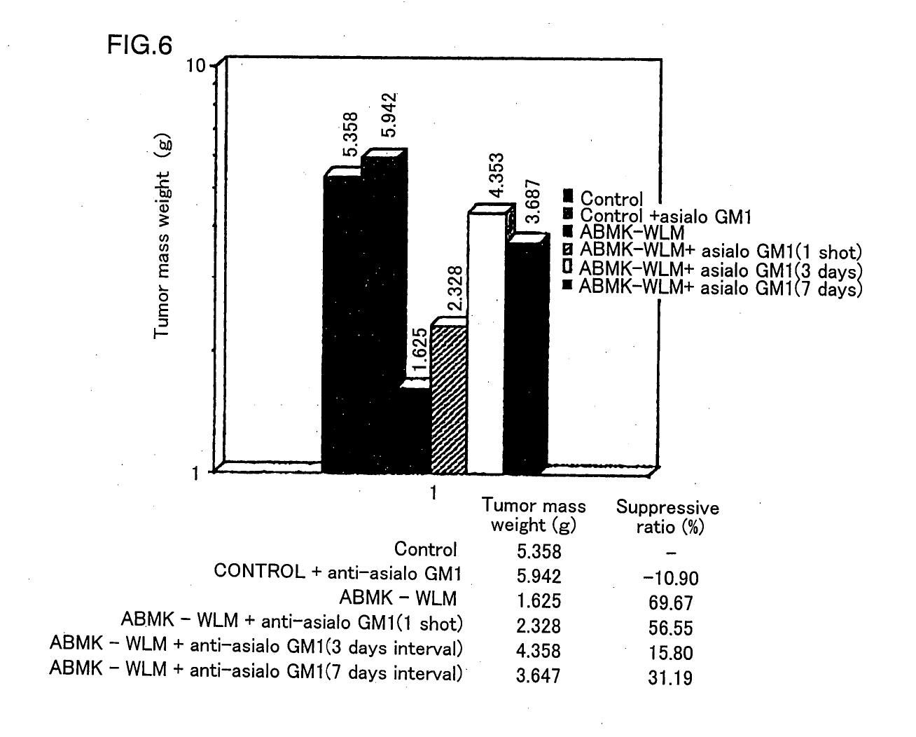

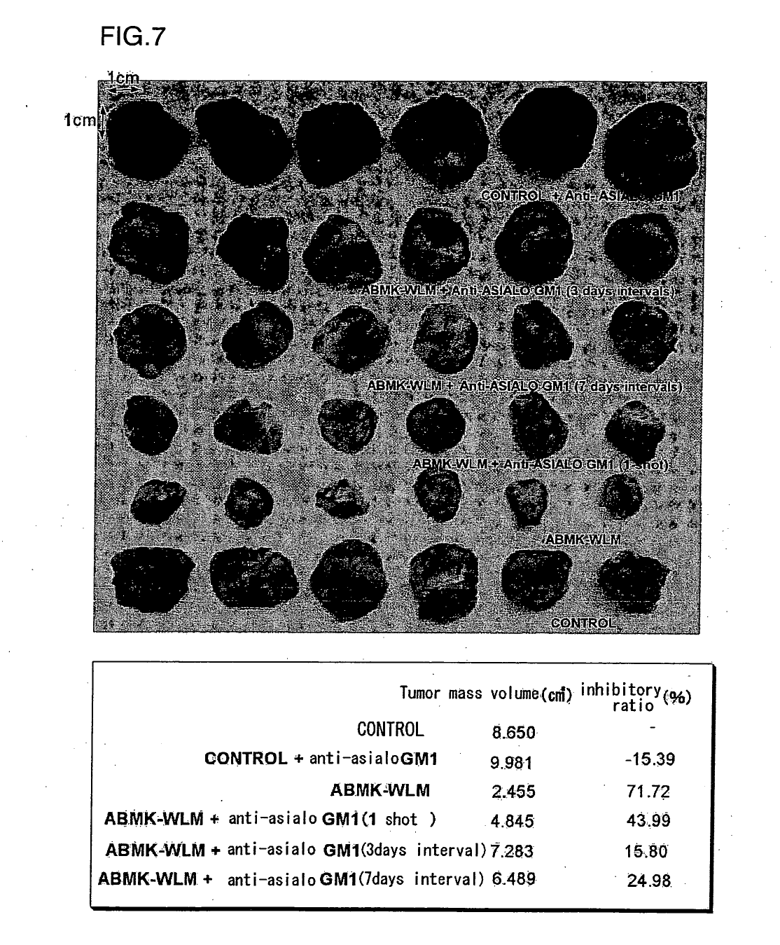

FIG. 6 indicates a comparison of tumor mass weights among each group of mice. FIG. 7 is a photograph comparing tumor sizes in each group of mice in the experiments shown in FIG. 4. In these figures, anti-asialo GM1 (FIG. 6) or anti-ASIALO GM1 (FIG. 7) abbreviates anti-asialo antibody treated mice.

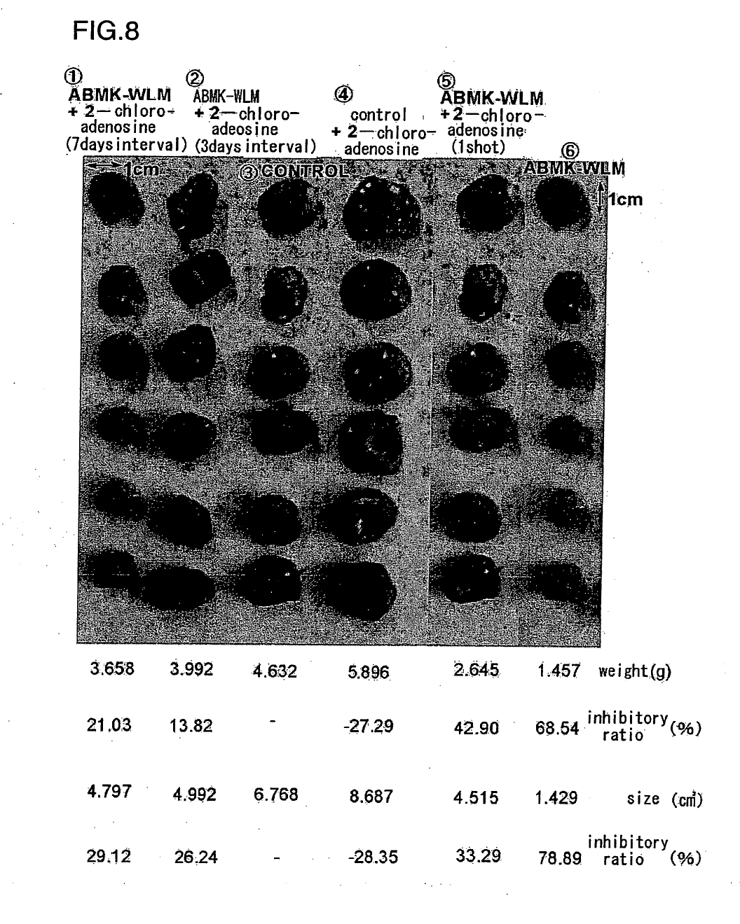

FIG. 8 is a photograph showing comparison of tumor weights and sizes among each group of mice in the experiments indicated in FIG. 5. In the figure, 2-chloroadenosine abbreviates 2-chloroadenosine treated mice.

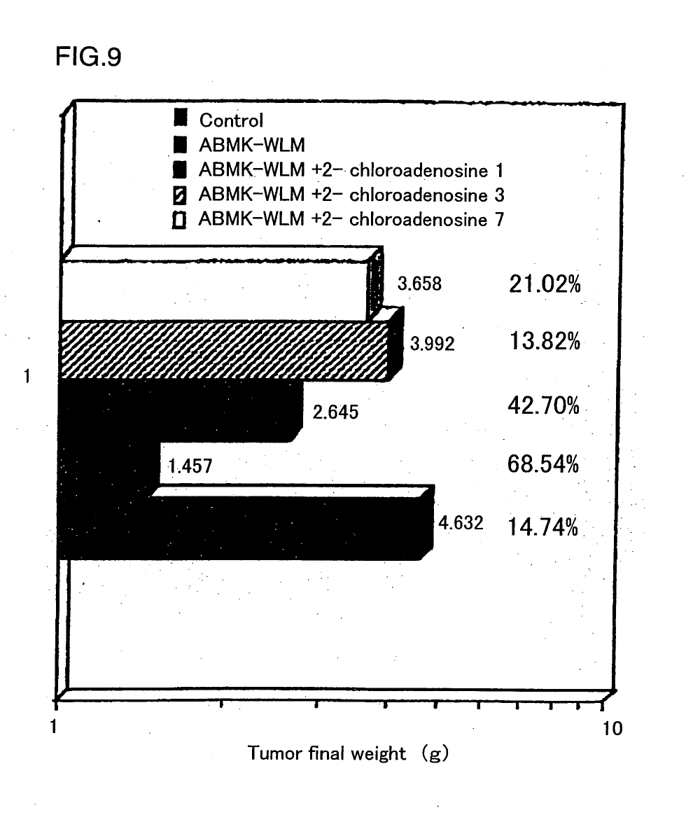

FIG. 9 is a drawing indicating a comparison among tumor mass weights at day 35 in mice of each group in the experiment in FIG. 5.

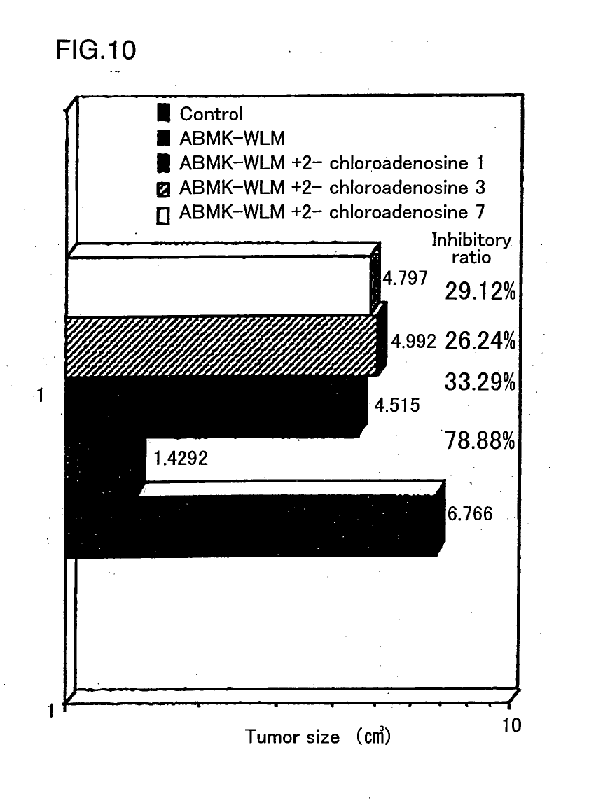

FIG. 10 is a drawing indicating a comparison among tumor mass volumes at day 35 in mice of each group in the experiment in FIG. 5.

As can be seen from these figures, strong tumor suppressive action of hot water extract of Kawariharatake and fractionated fraction thereof (Especially 1SY-16) was confirmed.

In summary, when cytotoxic activity was measured on KB cells, a mouse derived mouth cancer cell line, by using MTT reagent, the hot water extract of Kawariharatake and fractionated fraction thereof do not show significant cytotoxic activity (Results are not shown). When tumor suppressive effects were measured on implanted Sarcoma 180 using ICR/JCL-nunu mice (congenital T cells defective mice), oral administered hot water extract of Kawariharatake and fractionated fraction thereof do not show tumor suppressive effects. These results suggest that an immunological mechanism through T cells is involved in the tumor suppressive effects of Kawariharatake extract.

NK cells and macrophages are also important cell populations involved in the protection mechanism of the living body. When anti-asialo antibody, being selective in removing NK cells, and 2-chloroadenosine, being selectively toxic to macrophages, were used to inject mice through intra vein, it was observed that tumor growth was not suppressed, contrary to mice which are not treated by these drugs. Based on these results, it was suggested that Kawariharatake extract and fractionated fraction thereof, potentiate the immune system of the living body by activating these cell populations. It is considered that Kawariharatake extract and fractionated fraction thereof exert tumor growth suppressive effects through mechanisms such as activation of macrophages, tumor growth suppressive pathways through T cells, pathways in which macrophages exert anti-tumor action as acting as direct effector-cells, pathways exerting tumor killing action by activation of various immune responding cells and cytokines through NK cells.

### Example 3

### Study Regarding the Prolongation of Life by Kawariharatake Extract

1. Test Using Sarcorma 180

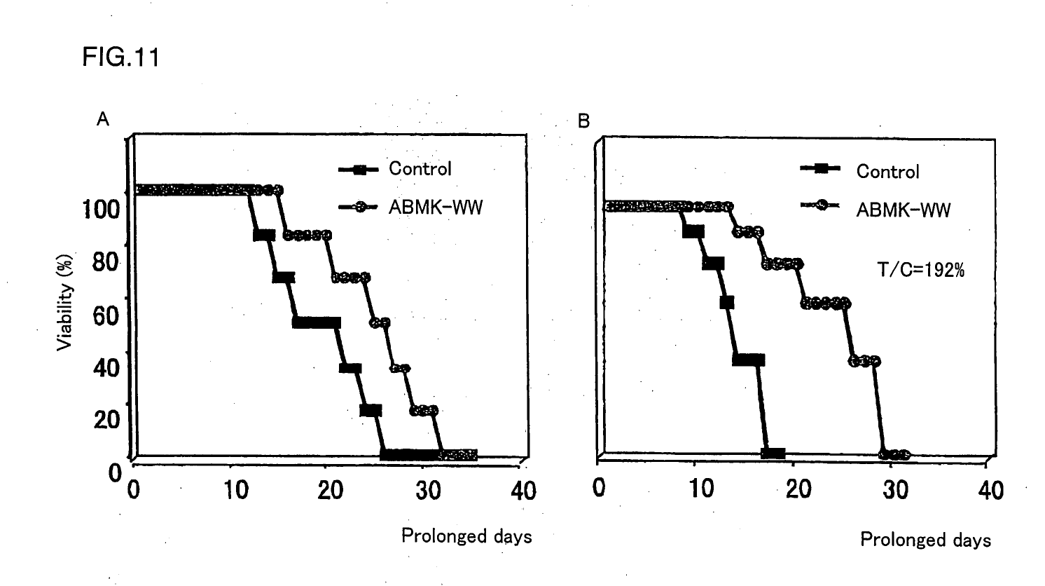

In order to examine the prolongation effects of the life of the mouse by Kawariharatake extract, Sarcoma 180 cells (1×10⁶ cells/mouse) were implanted intra peritoneally to 5 to 6 week old female ICR/JCL mice. Thereafter, Kawariharatake extract or fractionated fraction thereof was administered orally (100 mg as ABMK-WW/kg/day: administered by Sonde (probe or sound)), and viability was observed. The control group was administered distillated water similar to Example 2. Results are shown in FIG. 11A. In the figure, the horizontal line (X-axis) indicates the prolonged days of mouse host after implantation. The vertical line (Y-axis) indicates the % viability.

As shown in FIG. 11A, it was recognized that the test group to which Kawariharatake fractionated fraction was administered survived about 3 days longer than the control group. In the event that Sarcoma 180 cells were implanted subcutaneously, similar life prolongation effects can also be recognized (results are not shown).

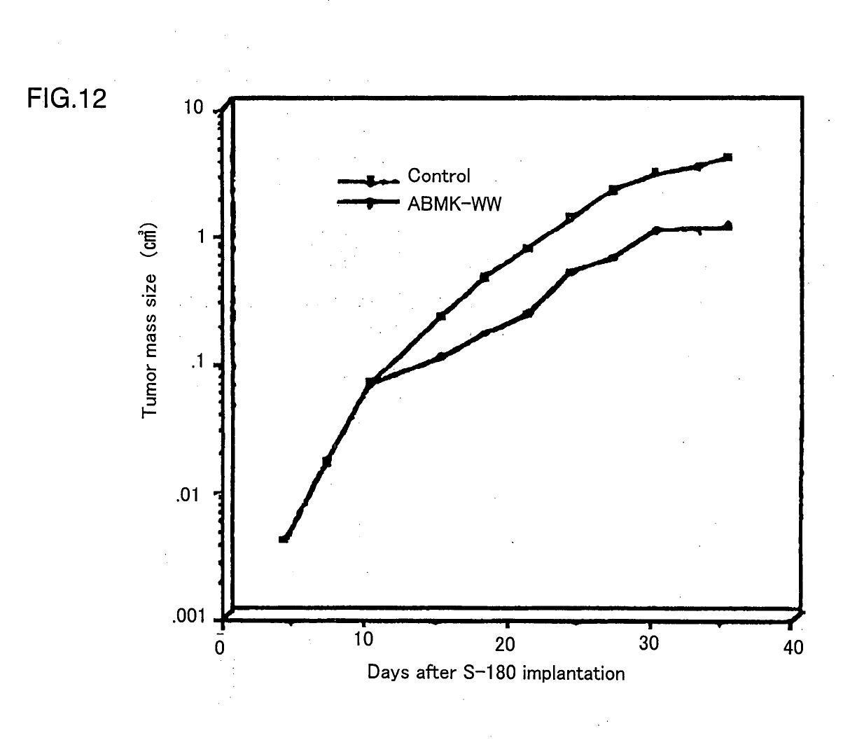

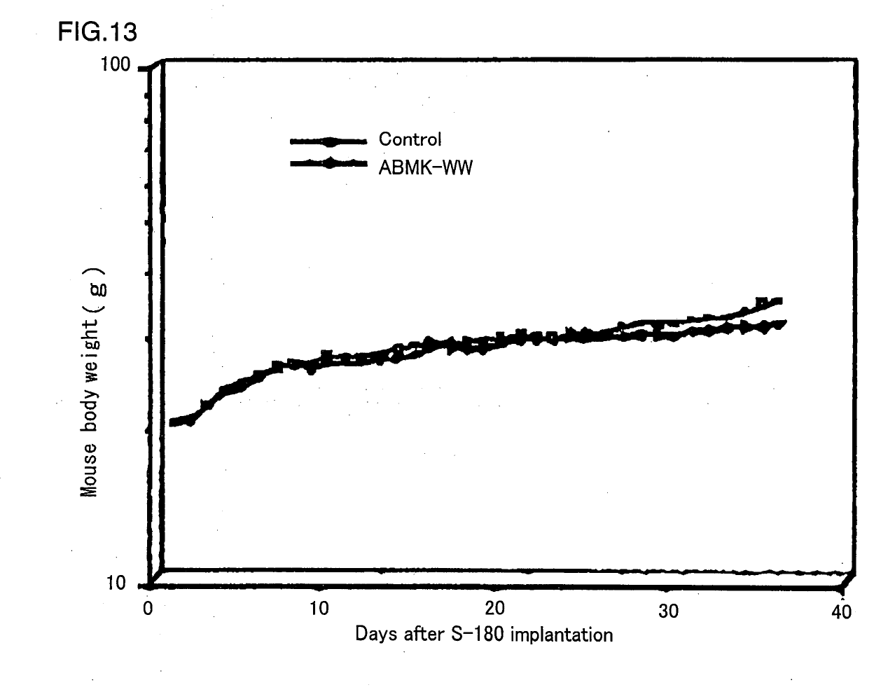

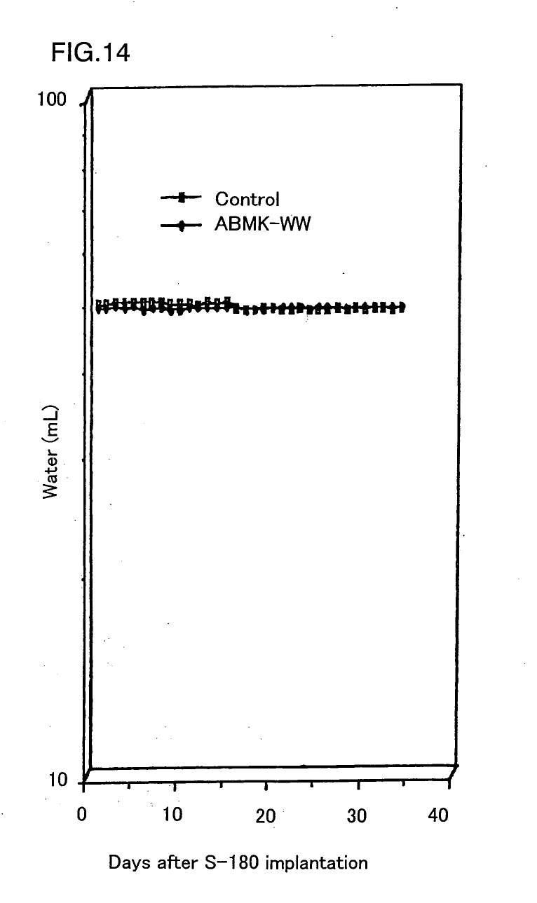

FIG. 11B shows results of similar tests as FIG. 11A, except that Kawariharatake fractionated fraction were ingested freely as drinking water, prepared by dissolving Kawariharatake fractionated fraction into the water of the water feeding tube (about 10 mg as ABMK-WW/kg/day). In this experiment, it was recognized that the test group to which Kawariharatake fractionated fraction was administered, survived about 10 days longer than the control group. FIG. 12 is a drawing indicating the transitions of tumor sizes in mice of each group in the experiment, in which mice ingested Kawariharatake fractionated fraction freely. FIG. 13 shows the transitions of body weight in the mice of each group. FIG. 14 is a drawing indicating the amount of fluid drunk by the mice of each group. As can be seen from the figure, the amount of fluid drunk by the mice of each group is almost the same.

Administration by Sonde appeared to give a stress to mice thereby shortening the prolongation in the Sonde administration group.

2. Test Using Meth A Fibrosarcoma.

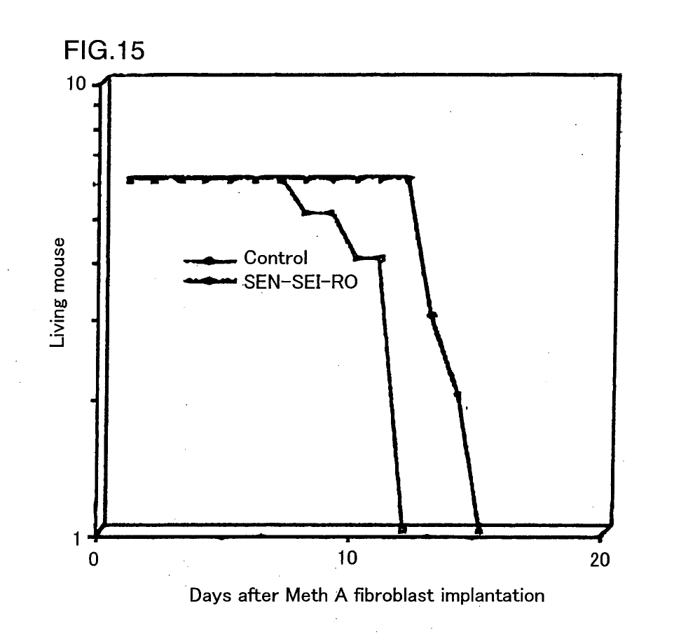

Meth A fibrosarcoma, which is a heterologous cell line, was used to examine the prolongation effects of life similar to the above 1. Meth A fibrosarcoma was injected subcutaneously. Results are shown as described in FIG. 15. About 3 days prolongation effects were observed in the Kawariharatake extract administered group. In the figure, results represented by SENSEIRO are results of the group administered the counterpart of AMBK-WW.

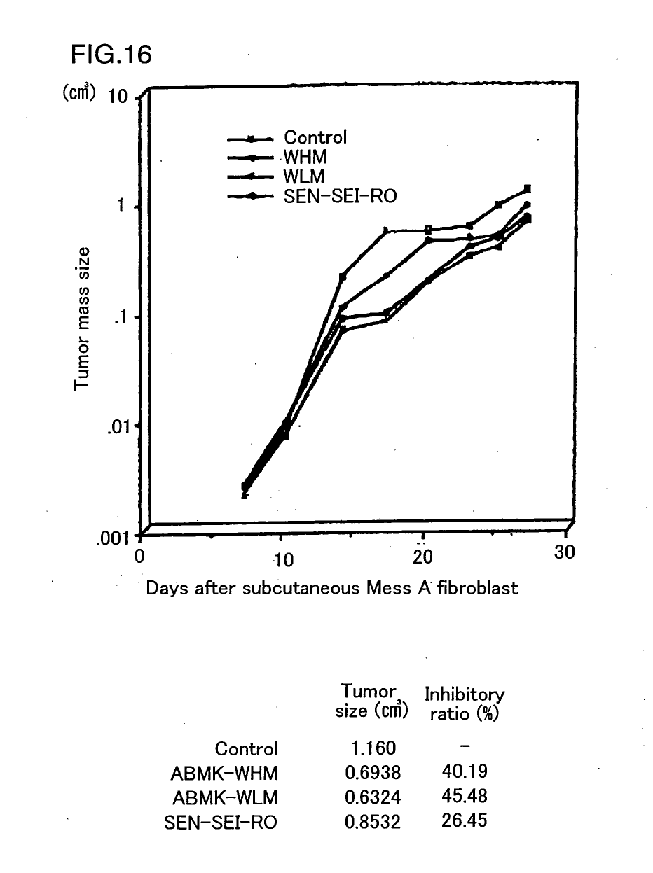

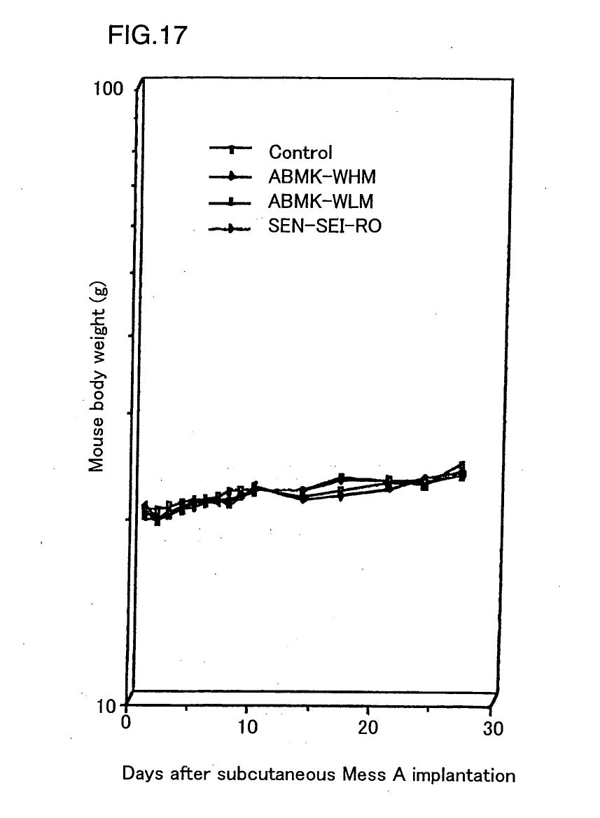

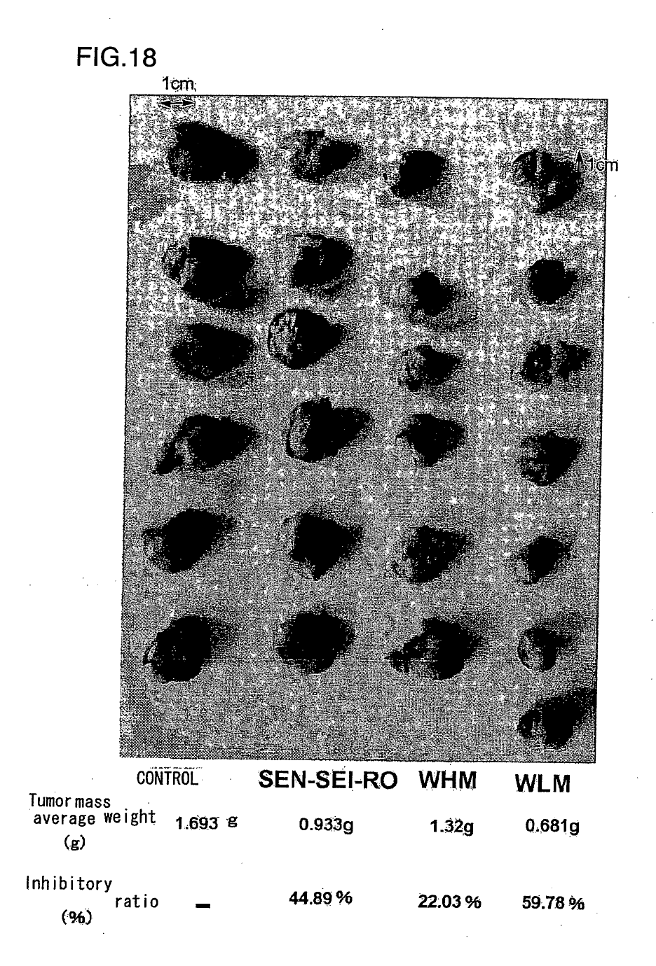

Then, similar assays were conducted with respect to tumor suppressive action by Kawariharatake extract or fractionated fraction thereof, similar to Sarcoma 180 as described above 1. Results are shown in FIGS. 16 to 18. FIG. 16 shows transitions of tumor mass volume in this experiment. FIG. 17 is a drawing indicating the transitions of body weights in each mouse. FIG. 18 is a photograph indicating the transitions of Meth A fibrosarcoma tumor sizes in each group. As shown in FIG. 18, it was recognized that Kawariharatake extract or fractionated fraction thereof also has tumor suppressive effects (22 to 60% inhibitory ratio), compared to mice of the control group, when Meth A fibrosarcoma is used.

### INDUSTRIAL APPLICABILITY

Low molecular weight ingredient (or faction) derived from Kawariharatake, material for drugs and food which are digested and absorbed well in vivo, and exerts a physiological activity through immune mechanism of the living body, are provided. Currently, cancer is a leading cause of death in Japan. As elucidating the relationship between cancer and the immune response of the host, immune therapy, which suppresses cancer growth and ultimately involute the cancer by increasing the immunocompetence of the host, is the focus of attention. As a means for activating the immune system of the host, there is a drug referred as BRMs (biological response modifiers). This drug is defined as a drug which makes cytokine and the like, directly to cancer as a specific or non-specific immune response adjusting factor, thereby changing the relationship between tumor and host. The composition of the present invention can be used alone or in combination therapy with chemical therapy drugs in use such as, improvement of QOL of cancer patient, enhancement of therapeutic effects of cancer drugs. The composition of the present invention can be used as material for an anti-cancer drug which has poor side effect.

1. A composition for exerting a physiological activity through the immune mechanism of the living body, comprising a hot water extract of Agaricus Blazei Muril.

2. The composition according to claim 1, wherein the immune mechanism is mediated by immunocompetent cells.

3. The composition according to claim 2, wherein the immunocompetent cells are selected from the group consisting of macrophages, T cells and killer cells.

4. The composition according to claim 2, wherein the immunocompetent cells are macrophages.

5. The composition according to claim 2, wherein the immunocompetent cells are T cells.

6. The composition according to claim 2, wherein the immunocompetent cells are killer cells.

7. The composition according to claim 1, wherein the physiological activity is a tumor-suppressive effect.

8. The composition according to claim 7, wherein the tumor is sarcoma.

9. The composition according to claim 8, wherein the sarcoma is Sarcoma 180.

10. The composition according to claim 7, wherein the sarcoma is Meth A fibrosarcoma.

11. The composition according to claim 7, wherein the physiological activity is the prolongation of life effect.

12. The composition according to claim 1, further comprising a pharmaceutically acceptable carrier.

13. The composition according to claim 1, being in the form selected from a group consisting of powder, liquid, tablet, capsule, and pellet.

14. A method for exerting a physiological activity through the immune mechanism of the living body comprising:
administering a composition to a patient, the composition comprising a hot water extract of Agaricus Blazei Muril.

15. A use of an extract of Agaricus Blazei Muril for preparing a composition which exerts a physiological activity through the immune mechanism of the living body.
